(19) United States
(12) Patent Application Publication
(10) Pub. No.: US 2003/0202965 A1
(43) Pub. Date: Oct. 30, 2003

(54) Methods and compositions for the preparation of cell transplants

(75) Inventors: Rainer Seubert, Waldkirch/Kollnau (DE); Eszter Tanczos, Freiburg (DE)

Correspondence Address: JONES DAY, 555 WEST FIFTH STREET, SUITE 4600, LOS ANGELES, CA, 90013-1025, US

(73) Assignee: BioTissue Technologies AG, Freiburg (DE)

(21) Appl. No.: 10/412,059

(22) Filed: Apr. 10, 2003

Related U.S. Application Data
(62) Division of application No. 09/731,580, filed on Dec. 6, 2000, now abandoned.

(30) Foreign Application Priority Data
Dec. 6, 1999 (DE) 19958693.4-41

(51) Int. Cl. A61K 45/00; A61N 1/30; C12N 5/08

(52) U.S. Cl. 424/93.7; 435/366; 604/20

(57) ABSTRACT

The present invention relates to a method for the cultivation of eucaryotic cells, the use of cells cultured according to said method for the preparation of in particular autologous, but also allogenous (cell) transplants, as well as in particular the use of accordingly prepared autologous melanocyte and/or keratinocyte transplants for the treatment of vitiligo patients; furthermore, also to melanocyte and/or keratinocyte transplants prepared accordingly. The present invention furthermore relates to new compositions comprising melanocytes as well as their use for the treatment of vitiligo patients; furthermore to the use of compositions comprising keratinocytes for the treatment of various dermatological symptoms and diseases.

This application is a divisional of U.S. patent application Ser. No. 09/731,580, filed Dec. 6, 2000, now pending. The foregoing application is hereby incorporated by reference as if fully set forth herein.

The present invention in general relates to the technical field of eucaryotic cell culture and tissue engineering. It specifically relates to a method for the cultivation of eucaryotic cells, the use of cells cultured according to this method for the preparation of in particular autologous, but also allogenous (cell) transplants, as well as, in particular, the use of autologous melanocyte and/or keratinocyte transplants prepared according to the present invention for the treatment of vitiligo patients; it furthermore also relates to melanocyte and/or keratinocyte transplants prepared according to the present invention. The present invention further relates to new compositions comprising melanocytes as well as their use for the treatment of vitiligo patients; furthermore, the use of compositions comprising keratinocytes for the treatment of various dermatological symptoms and diseases.

The medical-biotechnological field of tissue engineering encompasses the development, preparation, and modification of implants, biomolecules prepared in vitro, cells or tissues for the substitution or functional assistance of missing or injured body parts and tissues. One focus lies in the preparation of so-called autologous transplants. The underlying principle is to derive cells of the type to be transplanted from the healthy tissue of a patient, the so-called donor area, to culture them in vitro, to use them for the preparation of a transplant, e.g., by introducing them into a suitable carrier matrix, and to finally apply them to an accordingly prepared receptor area of the same patient. A decisive advantage of this kind of therapy lies therein that there is usually little danger of immunological responses or infection, which frequently occur in the case of heterologous transplants.

In principle, the preparation of such autologous transplants is imaginable on the basis of numerous cell and tissue types, e.g., skin, cartilage, bone, fatty or other kinds of tissue. In practice, the skin is, for example, particularly suitable due to its exposed location and thus easy accessibility. Furthermore, there are various dermatological indicational fields which require—often extended—skin transplantation. In this regard, in particular severe burn injuries as well as badly healing skin ulcera need to be mentioned on the one hand, on the other pigmentation deficiencies such as vitiligo.

Vitiligo is an acquired idiopathic, hypomelanotic change of the skin that is characterized by the loss of melanocytes within the skin and the hair bulb. Typical clinical symptoms are sharply delimitated depigmentated maculae (spots) of varying size, shape, and extension which may be localized on any part of the body. The prevalence of vitiligo lies about between 0.5 to 4%. Most frequently, the disease occurs in young people.

In spite of intensive research vitiligo remains a pigmentation deficiency of unknown etiopathogenesis. Accordingly, a causal treatment of vitiligo is as yet not possible. Present therapeutic approaches are, thus, limited to achieving repigmentation. In this regard one discriminates between operative and non-operative repigmentation measures. Adjuvant treatments such as camouflage or psychological guidance have proven to be a good approach, since the cosmetic disfiguration associated with vitiligo often result in psychosocial problems of those involved.

Common non-operative measures in vitiligo therapy are, for example, a combination therapy with psoralen and UVA-radiation, the so-called PUVA-therapy, UVB-radiation therapy, as well as the local or systemic corticosteroid therapy, respectively, optionally likewise in combination with UVA-radiation. The disadvantages of these conventional non-operative repigmentation measures are in particular the fact that patients as a rule respond to the PUVA-therapy only after 30 to 40 treatments and then to very differing extents, the phototoxic reactions induced by UVA as well as the common side effects associated with corticosteroid therapy.

Operative measures for the treatment of vitiligo often represent the therapy of choice, since conservative therapeutic measures very often remain without success; they may, if necessary, be applied in combination with a medicament and/or radiation therapy. In terms of operative repigmentation measures micro-tattooing as well as various autologous transplantation methods are the common ones to be mentioned. In the case of micro-tattooing ferric oxide pigments are implanted lesionally. In this context it is in particular problematic to achieve a satisfactory color harmonization with the surrounding skin, not least due to seasonal variations in skin color.

Autologous transplantation methods are far more relevant in practice and may be categorized essentially into the following four groups: (1) transplantation of autologous punch grafts of normal pigmented skin, which—as a rule—have a thickness of 1-2 mm, into a depigmentated receptor area (minigrafting); (2) transplantation of autologous cleft skin transplants; (3) transplantation of autologous suction blister transplants, as well as (4) transplantation of autologous skin cells, for example, melanocytes in the presence or absence of keratinocytes.

As regards the treatment of vitiligo patients by means of autologous transplantation of skin cells, the prior art discloses both the use of non-cultured as well as that of cultured autologous skin cells.

The first transplantation of autologous cultured melanocytes in a vitiligo patient was carried out 1987 by Lerner et al. (J. Invest. Dermatol., 1987, Vol. 89, pages 219-224).

Gauthier et al. (Journal of the American Academy of Dermatology, 1992, Vol. 26(1), pages 191-194) disclose that suspensions of non-cultured melanocytes and keratinocytes are particularly suitable for vitiligo therapy. However, a decisive disadvantage of such transplantations of non-cultured cells is that only a small amount of cells is available for transplantation.

Zachariae et al. (Acta Derm. Venereol., 1993, pages 46-48) disclose the successful transplantation of cultured melanocytes following transfer of the cells onto a collagen film, however only with a very small, statistically not significant number of patients. A transplantation of cultured melanocytes in combination with a carrier matrix characterized by plasticity or a gel-like consistency is not mentioned in the prior art.

In spite of the successes achieved with autologous skin cell transplants disclosed in the prior art, the focus—in regard of operative therapeutic measures for treating vitiligo—in practice still lies on autologous tissue transplantations. One reason for this is that such methods can be employed more easily and at a larger scale in routine practice, in spite of the fact that they often result in concomitant, undesired side effects, in particular from a cosmetic point of view. For instance, the danger of infection both in the donor as well as in the receptor area is largely diminished as compared to classical surgical transplantation methods, wound healing disorders occur less often, isolation of the autologous cells to be cultivated as well as their later transplantation occur virtually or completely painlessly, and the results are much more satisfactory also from a cosmetic point of view. Thus, various undesired side effect of the classic surgical transportation methods, for example, the “cobble stone effect” of the receptor side, keloid formation or hypertrophic scarring, are avoided by the transplantation of cultured cells.

In spite of that fact that these advantages were known and in spite of resulting need to extend the applicability of such autologous cell transplantations, it has as yet not been possible to develop a method for the cultivation of eucaryotic cells, in particular human cells, in particular for the purpose of preparing autologous and allogenous transplants, said method being suitable for routine application also at a larger scale and ensuring both the required safety and quality as well as the necessary efficiency. In particular, no according method for the preparation of transplants on the basis of eucaryotic, in particular human cells fulfilling the requirements of Pharmaceutical Law and other legal provisions such as, for example, the relevant EEC Directives has as yet been developed.

Thus, one of the objectives underlying the present invention was to provide a method enabling a routine culture of eucaryotic cells, in particular for the preparation of in particular, autologous, but also allogenous transplants that meets the official quality and safety requirements, in particular those of Pharmaceutical Law and the EEC Directives for the manufacturers of pharmaceuticals relating to a good manufacturing practice (GMP), thus enabling a utilization at a larger and possibly also at a commercial scale.

A further objective underlying the present invention was to provide new compositions for the treatment of vitiligo patients.

Another objective underlying the present invention was to provide methods for the treatment of further dermatological symptoms and diseases.

It was found that a routine culture of eucaryotic, in particular human cells for the preparation of in particular autologous, but also allogenous (cell) transplants meeting the official quality and safety requirements is possible in the event that conditions that are in accordance with the requirements according to sterility class “A” according to the GMP Regulations are complied with. An application of such a clean-room technology in the field of tissue engineering per se, and in particular for the preparation of human transplants, is unknown to the present Applicant.

Accordingly, the present invention relates to a method for the cultivation of eucaryotic cells characterized in that steps in which the cell cultures are open, i.e., come into contact with the surrounding air, are carried out under conditions which comply with the requirements of the sterility class “A” according to the GMP Regulations. The method for the cultivation of eucaryotic cells according to the present invention is in particular characterized in that the steps of the method in which the cell cultures are open, i.e., come into contact with the surrounding air, are carried out in a closed system, i.e., in a system hermetically sealed off against the surroundings, and at overpressure. Thus, the method of cultivation according to the present invention differs both from the conventional methods for the cultivation of eucaryotic cells, in which the steps in which the cell cultures are open are carried out in an open system at overpressure, as well as from the closed systems used, for example, in the fields of infection research or transplantation medicine (e.g., in connection with bone marrow transplantations), in which under-pressure is prevailing and which require whole body-masking.

In a preferred embodiment according to the present invention said steps are carried out in working insulators meeting the requirements of the sterility class “A” according to the GMP Regulations, i.e., under virtually aseptic conditions. Technically, this level of sterility is ensured by several factors, in particular: overpressure within the insulator and laminar air flow, as well as the application of suitable devices, for instance suspended particle filters, e.g., by means of a HEPA (High efficiency particular air filtration) filter suitable for sterility class “A”, by means of which 99,99% of all particles larger than 0,3 μm can be removed, as well as by channeling in disinfected materials, for example, from a class “D”-clean room.

The specification of the sterility classes in connection with the present invention concurs with the requirements in force at the time point of the filing of the application according the EEC-Manual for a Good Manufacturing Practice of Pharmaceuticals (cf., for example, “EG-Leitfaden einer Guten Herstellungspraxis für Arzneimittel—mit Betriebsverordnung für pharmazeutische Unternehmer [=EEC-Directive for Good Manufacturing Practice for Pharmaceuticals—Including an Operating Regulation for Pharmaceutical Manufacturers]” (1998), 5^{th}, revised and extended edition, Ed.: G. Auterhoff, Editio Cantor Verlag Aulendorf). Therein, the first supplementary and revised guidelines for the Preparation of Sterile Pharmaceuticals in the Annex to this EEC-Manual comprises definitions of the sterility classes “A”, “B”, “C” and “D”, according to which the quantitative classification occurs according to the number of particles present in the air.

As regards sterility class “A”, it is stated that not more than 3500 particles larger than at least 0.5 μm and 0 particles that are larger than at least 5 μm may be present per cubic meter of air. The recommended (average-) threshold values for microbiological contamination in sterility class “A” lies—for the operational state, i.e., the state in which the facility is operated for its intended purpose and with the number of personal listed—constantly at<1 cfu/m³, i.e., in the air sample; for petri dishes with a diameter of 90 mm over a time period of 4 hour; for contacting dishes with a diameter of 55 mm; and for a 5 finger-glove print (cf., ibid., in particular, pages 73-77).

In a preferred embodiment the sterility class “A” insulators are located within sterility class “D”-surroundings. Said insulators consist of permanently joined transfer- and working- insulators. The GMP areas have to be equipped with according ventilation systems. Preferably an approximately 20-fold change in air per hour is ensured. According to this preferred embodiment, the processed air passes over a suspended particle filter—belonging to the class S2-HEPA-filters—via outlets and is subsequently blown back into the sterility class “D” GMP-area. As mentioned above, the capacity of such a filter lies at more than 99.997% for a particle size of 0.3 μm.

During the actual production phase the working insulators can only be loaded via the transfer insulators, which—in connection with the present invention—preferably belong to sterility “B”. The transfer insulators are likewise equipped with an according ventilation system so that the air is continuously exchanged via a filter also in said transfer insulators. In order to ensure an aseptic channeling-in of materials into the working insulators, loading of the transfer insulators is only possible in the event that the working insulators are closed. Both the working as well as the transfer insulators are sterilized every month via hydrogen peroxide fumigation. The remaining clean-room surroundings of said working and transfer insulators may belong to a lower sterility class, e.g., the sterility classes “B”, “C” or “D”. According to a preferred embodiment of the present invention those cultivation steps or steps for the preparation of an autologous or allogenous (cell) transplant in which the cell cultures are not open are carried out in the clean-room surroundings of said working and transfer insulators.

In said clean-room area surrounding the isolator, which according to a preferred embodiment of the present invention belongs to sterility class “C” or “D”, a permanent overpressure—as compared to the normal atmospheric pressure—has to be ensured. An overpressure of more than about 5 to 10 Pascal is preferred. An even larger pressure gradient exists between the sterility class “A” working insulators and the lower sterility class surroundings, preferably an according overpressure of larger than about 25 Pascal, preferably of larger than about 50 Pascal, is adjusted in the sterility class “A” working insulator.

Thus, there exists both a pressure barrier as well as—due to the construction of the insulator—a physical barrier, the latter consisting, for example, of plexiglass, between the working insulator in which the cultivation or preparation steps, respectively, in which the cell cultures are open, are carried out under sterility class “A” conditions on the one hand and the surroundings, in which there lower sterility class, preferably class “C” or “D” conditions prevail, on the other.

In a preferred embodiment of the present invention the working and transfer insulators belonging to sterility class “B” consist, for example, of stainless steel with inner edges that are rounded off (concave moulding) and are glove systems. In principle, however, both the working and the transfer insulators as well as the gloves used may, of course, consist of any material fulfilling the desired purpose.

According to the present invention all steps to be carried out in the course of the cultivation and/or the preparation of (cell) transplants, i.e., both those in which the cells are “open”, i.e., in contact with the surrounding air, as well as those in which the cells are not in such contact, for example, during the incubation times in the incubator or during centrifugation, may, of course, be carried out under sterility class “A” conditions. The only prerequisite therefor is that the required apparatus, etc. can be introduced into the clean room of said sterility class and can be put in operation.

The method according to the present invention is in principle suitable for the cultivation of any type of eucaryotic cells, in a preferred embodiment in particular human cells are cultivated according to this method. Said method is furthermore particularly suitable for the cultivation of human cells in the context of the preparation of autologous and allogenous (cell) transplants, for example, for the treatment of various tissue deficiencies or in the case of organ failure. Thus, the present invention also encompasses an according cultivation for the preparation of three-dimensional transplants, for example for the preparation of three-dimensional skin, bone, or cartilage substitutes. In the case of such transplants the shape of the tissues to be transplanted may also be defined. For the sake of illustration only a three-dimensional joint substitute, a three-dimensional finger bone substitute, or a three-dimensional ear substitute should be mentioned by way of example.

In the context of the present invention skin cells, in particular, melanocytes, keratinocytes, fibroblasts as well as bone cells, cartilage cells, adipocytes, vessel cells, cells of the oral mucous membrane, urothelial cells, muscle cells, cells of the nervous system, haematopoetic cells, tendon cells, other cells of the motility system, embryonic or other stem cells, liver cells, kidney cells, heart muscle cells, epithelial cells of various mucous membranes as well as hormone- or transmitter-producing cells, for example, pancreatic insular cells, glandular cells, corneal epithelial cells, etc., are preferably cultivated according to the method according to the present invention. Particularly preferred are skin cells, in particular melanocytes, keratinocytes, and fibroblasts, as well as bone cells, cartilage cells, adipocytes, vessel cells, cells of the oral mucous membrane, urothelial cells, muscle cells and cells of the nervous system.

The parameters and conditions that need to be complied with when culturing eucaryotic cells (the composition of the culture media, temperature, CO₂-content, etc.) are in principle known to the skilled person, likewise additional methods which have proven to be particularly advantageous when cultivating eucaryotic cells. By way of example, “spinner” culture techniques are mentioned here in which the cells are cultured on so-called microcarriers, i.e., small beads consisting of suitable, for example, resorbable biological or synthetic methods, by means of which a large surface expansion is achieved.

As already mentioned, (cell) transplants, and in particular autologous, but also allogenous human (cell) transplants, may be prepared according to the method of cultivation according to the present invention. As will be explained in more detail in the following these (cell) transplants may optionally comprise suitable carrier substances.

As already mentioned above it is in principle possible in many cases to culture eucaryotic cells derived from a tissue and to use said cells for the preparation of autologous, in some cases also allogenous transplants, for example, for the treatment of tissue deficiencies or in the case of organ failure.

The principle approach is briefly shown by means of the following two examples, which will be explained in somewhat more detail in the following:

(i) preparation of a joint substitute comprising deriving a piece of cartilage, cultivating the cartilage cells and combining them with suitable synthetic or biological matrix material;

(ii) preparation of a piece of bone comprising deriving a piece of bone or a bone marrow biopsy, cultivating the bone cells (for example, bone cell precursors or mature bone cells) and combining them with suitable synthetic or biological matrix materials.

The above makes it clear that an according approach is in principle imaginable for all eucaryotic or human cell types, respectively.

In a preferred embodiment according to the method of the present invention, human melanocytes and keratinocytes, possibly in combination with fibroblasts and/or other (skin) cells, as well as possibly also in the context of a three-dimensional skin culture, are cultured for the purpose of preparing autologous human transplants. The preparation of such melanocyte and/or keratinocyte transplants utilizing the method of cultivation according to the present invention particular preferably comprise—either after or during the cultivation of the according skin cells—a step in which the cells are brought into a suitable carrier matrix. This is explained in more detail in the following.

In such cell cultures or transplants based upon skin cells, respectively, the ratio between melanocytes and other cell types present, for example, keratinocytes, is uncritical; likewise the sole presence of only one cell type, for example, of melanocytes or keratinocytes, respectively, is imaginable and possible.

As mentioned above, the method of cultivation according to the present invention is in particular characterized in that it meets the highest safety requirements and is, thus, suitable for the preparation of autologous or allogenous (cell) transplants. Thus, a method according to which the preparation of such transplants may be carried out routinely and at a larger scale and in compliance with official safety and quality requirements is for the first time available to the skilled person.

Accordingly, the present invention further relates to the use of eucaryotic cells cultured according to the method of the present invention as autologous transplant or for the preparation of an autologous transplant or a three-dimensional tissue substitution material. The method according to the present invention may in principle be used for the preparation of all autologous transplants based upon cultured eucaryotic cells. Preferably the method according to the present invention is used for the preparation of autologous transplants comprising skin cells, in particular melanocytes, keratinocytes and/or fibroblasts, bone cells, cartilage cells, adipocytes, cells of the oral mucous membrane, vessel cells, cells of the nervous system, muscle cells, or urothelial cells.

The present invention further relates to transplants resulting from a preferred embodiment of the present invention, namely transplants comprising melanocytes and/or keratinocytes characterized in that said melanocytes and/or keratinocytes are cultured according to the method according to the present invention.

In the context of hormone- or transmitter-producing cells it is imaginable to utilize also allogenous cells that are transplanted in capsules which enable the release of the produced or secreted molecules, respectively, and which at the same time protect the allogenous cells from immunological responses. Such capsules are, according to their function, as a rule made of biologically non-degradable, non-immunogenic material. Depending on the target tissue involved, various methods of application are imaginable and possible, for instance, endoscopic or subcutaneous application. The size of such capsules is by and large uncritical and preferably lies within the micro- to millimeter range. The release of the molecules produced inside these capsules or secreted, respectively, in the most simple case occurs via diffusion. The material of which said capsules consist, thus, preferably exhibit a (fine) porous structure enabling such a release.

The present invention accordingly relates also to the use of hormone- or transmitter-producing cells cultured according to the method of the present invention in combination with suitable capsules as allogenous transplants or for the preparation of allogenous transplants.

According to the method of the present invention the eucaryotic cells to be transplanted are, after they have been taken from the donor tissue, cultured under the conditions defined above (sterility class “A” at least during the culture or preparation steps, respectively, in which the cell cultures are open) and are subsequently—if desired—taken up in a suitable matrix under said conditions or brought into a shape suitable for transplantation in another way or prepared for use in the context of a treatment of one or more tissue deficiencies or of organ failure.

The following are intended to illustrate this: Transplants made of suspended cells in which said cells are preferably present in a gel-like, injectable material; cell sheets, i.e., monolayer cellular structures applied to a suitable, for example, membrane-like carrier material; as well as three-dimensional transplants within which the cultured autologous cells are brought into a defined, three-dimensional shape in conjunction with a suitable matrix, for example, the shape of an ear or a joint.

In particular in the context of the preparation of autologous transplants comprising skin cells, in particular, melanocytes and/or keratinocytes, bone and/or cartilage cells, optionally in combination with further cells, it has been observed that it is often advantageous if the cells are transplanted in combination with a suitable matrix as carrier material. Accordingly, such cells are often brought into an according carrier material during or after cultivation, but prior to transplantation.

A matrix suitable for this purpose may in principle be based either upon biological molecules which in vivo occur as components of the extracellular matrix (ECM), such as collagens, hyaluronic acid esters, and glycosaminoglycan esters, or on derivatives of such ECM molecules, furthermore on fibrin or fibrin derivatives as well as on suitable synthetic carrier materials. As a rule, biologically degradable materials are preferred.

Apart from this, also specific applications are in principle imaginable, for instance in the context of the already mentioned use according to the method of the invention of cultured hormone- or transmitter-producing cells in combination with suitable capsules as allogenous transplants or for the preparation of allogenous transplants, in the case of which a use of biologically non-degradable materials could also be advantageous.

In the method according to the present invention for preparing autologous, melanocyte and/or keratinocyte comprising transplants a carrier matrix explained in more detail in the following is preferably used, said carrier matrix being characterized in that it exhibits plasticity or a gel-like consistency.

An especial advantage of said plasticity or gel-like consistency, respectively, lies therein that the according transplants are particularly suitable for a transplantation to problematic receptor areas, for example, on the hands, the neck (back and front), or within the genital region, i.e., those areas which as a rule are strongest affected by vitiligo.

Therefore, in the method according to the present invention for the preparation of autologous, melanocyte and/or keratinocyte comprising transplants, a carrier matrix comprising collagen gel, fibrin glue and one or more other fibrin derivatives, hyaluronic acid derivatives or hydrogels or mixtures thOreof is preferably utilized, particularly preferred is a carrier matrix comprising fibrin glue and/or one or more (other) fibrin derivatives.

In the context of bone transplants the utilization of a human spongiosa is particularly suitable, i.e., of an autoclaved or otherwise disinfected bone substance based upon human bones, which, however, does not anymore contain bone cells. The best results may be achieved in the event that bone cells are cultured in the presence of a human spongiosa or are combined with a human spongiosa after cultivation. In principle also other carrier matrices are imaginable in combination with bone transplants, for example biomolecules representing components of the extracellular matrix or also suitable synthetic materials, preferably, however, such molecules and/or materials that exhibit a porous structure. Examples therefor are hydroxyapatite, calcium phosphate and collagens. Furthermore, an injectable bone transplant, preferably on the basis of a matrix exhibiting plasticity or a gel-like consistency, for example on the basis of fibrin, is likewise imaginable.

Also in connection with the preparation of autologous cartilage transplants according to the present invention the skilled person is by no means limited with respect to the choice of suitable carrier materials. In the context of the present invention gel-like and/or injectable forms of transplants are preferred also in combination with cartilage cells (chondrocytes), said transplant forms comprising a carrier substance based upon one or more collagens, fibrin or fibrin derivatives, hyaluronic acid derivatives or hydrogels or mixtures thereof.

In the course of the studies underlying the present invention it was found that in particular autologous skin cell transplants comprising cultivated autologous melanocytes either alone or in combination with keratinocytes in a carrier matrix exhibiting plasticity and gel-like consistency exhibit particularly good transplantation properties both in animal experiments and in vitiligo patients. In particular, good therapeutic results can be observed in the event that the acceptor area affected by vitiligo is subjected to a suitable pretreatment, in particular, if it is, for example, abrasure by ER-YAG laser prior to transplantation. Subsequently, the melanocytes cultured according to the present invention are brought into a suitable carrier matrix, for example fibrin glue, and are applied to said area, which is then bandaged. As a rule, the primary bandage can be removed after approximately a week, the transplanted sites are preferably subjected to UV-radiation about once a week. In this regard, a UVB- or a UVA-radiation is, for example, in principle possible, for instance in the context of a PUVA-therapy. A preferred embodiment comprises a UVB-radiation at a wave length of 311 nm over a suitable time period. 100551 It goes without saying that a combination with further possible advantageous therapeutic measures is not excluded. In the case of a therapy according to the above example the first repigmentation processes may often be observed already after a few weeks.

Specifically in the context of a transplantation of cultured, in particular autologous melanocytes of keratinocytes a further use—in addition to the already explained vitiligo therapy—is imaginable in connection with repigmentation, papilla-imitation or breast-rebuild, for instance following a total or subtotal breast amputation. For this purpose cultured, in particular autologous melanocytes alone or in combination with other, as rule likewise cultured cell types, for instance keratinocytes, fibroblasts, chondrocytes, cells of the nervous system as well as vessel cells may be used. As regards breast-rebuilding, it will of course make sense to do this in the context of or in combination with other methods known to the skilled person, for example, a free tissue transfer or a rebuild using silicon implants.

Skin cell transplants prepared according to the method of cultivation according to the invention are characterized not only by the general advantages of such transplants based upon autologous cells listed above, but in addition also exhibit additional advantages.

In this context it should once more be specifically mentioned that the plasticity and gel-like consistency of the transplants preferred in the context of the present invention are particularly suitable for an application at sites like, for example, the hands, the neck (back and front) as well as the genital region, the treatment of which was problematic with the methods disclosed in the prior art. However, these are the sites at which vitiligo as a rule occurs most frequently. Due to said plasticity the cells are transplanted more effectively and their subsequent adhesion, proliferation and migration is stimulated, which in turn results in a better “take”, i.e., an improved integration of the transplant. The general rule is that the larger the “take”, the larger also the chance of an optimal repigmentation.

In summary, it can thus be stated that skin cell transplantations in connection with the methods and compositions according to the present invention disclosed herein are—for example, but by no means exclusively in the context of vitiligo therapy—not only much simpler to carry out as compared to other methods, but in addition also significantly promote the success of the transplantation or the repigmentation in question.

Finally, it was also found that autologous skin cell transplants comprising cultured autologous melanocytes and/or keratinocytes in a carrier matrix exhibiting plasticity and a gel-like consistency also exhibit very good transport and application properties. For example it was shown that autologous melanocytes in a preferred embodiment of the compositions according to the present invention, which are explained in more detail in the following, as a rule exhibit a vitality of up to 80% even after a transport or storage at room temperatures for 24 hours.

Accordingly, the present invention also relates to compositions comprising melanocytes in a carrier matrix, preferably in a carrier matrix exhibiting plasticity or gel-like consistency, as well as the use of such compositions as autologous transplants for the treatment of vitiligo patients. According to the above the use of cultured melanocytes is preferred, this notwithstanding, however, a use of according non-cultured skin cells is likewise imaginable.

If cultured melanocytes are used in connection with the compositions according to the present invention mentioned above, then there exists the possibility to culture said melanocytes according to the method of the present invention disclosed herein. However, the skilled person will understand that in connection with said compositions according to the present invention comprising cultured melanocytes in a carrier matrix exhibiting plasticity or gel-like consistency cultivation may, in principle, be carried out according to any method of cultivation suitable for this purpose.

In the compositions according to the present invention comprising melanocytes in a carrier matrix exhibiting plasticity or a gel-like consistency melanocytes may represent the only cell type, however, they may also be present in combination with other (skin) cell types, for example, keratinocytes and/or fibroblasts. In the latter case, the ratio between melanocytes and further cell types present, for example, keratinocytes, is uncritical.

A suitable carrier matrix may, in principle, be based upon either biomolecules that are present in vivo, for example, as components of the extracellular matrix (ECM), for example, collagens, hyaluronic acid esters and glycosaminoglycan esters or on derivatives of such ECM molecules, furthermore, on fibrin or fibrin derivatives as well as on suitable synthetic materials. As a rule, biologically degradable materials (“biomatrices”) are preferred, particularly preferred are biomatrices on the basis on fibrin glue and/or other fibrin derivatives.

Fibrin glues represent biological two component-carrier systems consisting essentially of a fibrinogen and a thrombin component. Coagulation, i.e., the transformation of fibrinogen to fibrin, is induced by the addition of thrombin. The extent of coagulation and thus the consistency of the carrier system can be adjusted according to the thrombin concentration chosen. A detailed disclosure of the carrier system fibrin glue, its preparation, its use for the culture of keratinocytes as well as the use of such keratinocyte cultures as transplants may be found in EP 0 373 044, the according contents of which are enclosed in the present application by reference in their entirety.

The compositions preferred in the context of the present invention, comprising autologous melanocytes in a carrier matrix based upon fibrin glue exhibit the further advantage that they can quite easily be subjected to histological analyses for the detection of melanocytes. For this purpose, the melanocyte suspension which is initially present in the fibrinogen solution of the fibrin glue is mixed with the second component of the fibrin glue, i.e., a thrombin solution, in a so-called clotting-step. Thereby, the formation of fibrin is induced, a so-called clot is formed. Said clot is then shock-frozen, for example, on dry ice or in liquid nitrogen, and is used for the preparation of frozen sections. The thus obtained frozen sections can then be stained by means of immunohistological methods well-known to the skilled person by using a suitable antibody, for example, the commercially available HMB 45-antibody (DAKO), which is specific for melanocytes.

In the course of the studies underlying the present invention it was shown that skin cells, in particular, melanocytes and/or keratinocytes, that are transplanted in the form of the compositions according to the invention, i.e., in a carrier matrix, adhere to the accordingly pretreated tissue of the acceptor area, proliferate and migrate rapidly. Thereby, more rapid therapeutic successes are often achieved as compared to the melanocyte transplants conventionally utilized in vitiligo therapy, without the quality or the durability of the repigmentation or the cosmetic result, i.e., due to scare formation being adversely affected.

Accordingly, the present invention further relates to the use of the compositions according to the present invention described herein comprising melanocytes in a carrier matrix or of compositions comprising melanocytes and/or keratinocytes cultured according to the method according to the present invention disclosed herein as autologous transplant or for the preparation of an autologous transplant for the treatment of vitiligo patients.

In this event, transplantation occurs according to methods well-known to the skilled person which may likewise optionally comprise a pre- and/or posttreatment of the area to be treated, for instance, by means of a laser pretreatment for the purpose of abrasion of the tissue, for instance, by means of a Er-Yag laser and/or a UV-radiation.

In addition, it was surprisingly found that compositions comprising keratinocytes in combination with a carrier matrix characterized by plasticity or gel-like consistency may be utilized in an extremely advantageous manner in the treatment of various dermatological symptoms or diseases, respectively, for instance in the treatment of (a) solar pre-cancerous forms, (b) pregnancy-induced changes in skin (e.g., chloasma), (c) rosacea and rhinophyn (“lumpy nose”), (d) age-induced changes in skin (e.g., formation of wrinkles), (e) keloids, hypertrophic scares, acne scares as well as therapy-resistant acne.

A carrier matrix suitable for this purpose can, in principle, be based upon either biological molecules which occur in vivo, for example, as components of the extracellular matrix (ECM), such as collagens, hyaluronic acid esters and glycosaminoglycan esters, or on derivatives of such ECM molecules, furthermore on fibrin or fibrin derivatives as well as on suitable synthetic carrier materials. As a rule, biological degradable materials are preferred, i.e., again biomatrices are utilized preferably with biomatrices on the basis of fibrin glue and/or other fibrin derivatives are particularly preferred.

Given the above, the skilled person will understand that also in this case the cultivation of the skin cells, i.e., of keratinocytes alone or in combination with other cell types, may in principle occur according to all methods for eucaryotic cell cultivation known to the skilled person, including the method of cultivation according to the present invention disclosed herein.

Accordingly, the present invention also relates to the use of compositions comprising keratinocytes as autologous transplant or for the preparation of an autologous transplant for the treatment of (a) solar pre-cancerous forms, (b) pregnancy-induced changes in skin (e.g., chloasma), (c) rosacea and rhinophyn (“lumpy nose”), (d) age-induced changes in skin (e.g., formation of wrinkles), (e) keloids, hypertrophic scares, acne scares as well as therapy-resistant acne.

Here again, transplantation is carried out according to methods known to the skilled person, optionally comprising a pre- and/or post-treatment of the area to be treated, for example by means of a laser pretreatment for the purpose dermabrasion, for example, by means of an ER-YAG-laser and/or a UVB-radiation treatment.

The following example serves to explain the present invention in more detail, but is by no means intended to limit it.

A biopsy taken from a patient is, after desinfection, transferred to surroundings in which the conditions of sterility class “A” according to the EEC-Directive for a good manufacturing practice for pharmaceuticals are fulfilled, for example, via a transfer insulator into a working insulator, in both of which virtually absolute sterility prevails.

In the case of a cultivation of melanocytes the following steps are carried out, preferably under the sterility conditions given in brackets:

Final preparation of an autologous melanocyte transplant:

Cells not taken up in thrombin solution are cryo-conserved either immediately or after further cultivation until subconfluency.

Prior to application of the autologous transplant the vitiligo area is subjected to abrasion using an ER-YAG laser. The suspension of the autologous culture melanocytes in fibrin tissue is applied to the according sites. The sites are bandaged, the primary bandage is removed after one week. After that, UV-radiation is carried out once a week (UVB, 311 nm, over a time period of usually at least 12 weeks). Normally, a repigmentation of the treated skin areas sets in after a few weeks.

We claim:

1. A method of cultivating eucaryotic cells comprising cultivating eucaryotic cells under an open state wherein the open state is under conditions with the requirements of sterility class “A” according to the GMP Guidelines.

2. The method according to claim 1, wherein said open state is a closed system at an overpressure.

3. The method according to claim 1, wherein said eucaryotic cells are cultured human cells.

4. The method according to claim 2, wherein said eucaryotic cells are cultured cells selected from the group consisting of skin cells, bone cells, cartilage cells, adipocytes, vessel cells, cells of the oral mucous membrane, urothelial cells, muscle cells, cells of the nervous system, haematopoetic cells, tendon cells, other cells of the motility system, embryonic and other stem cells, liver cells, kidney cells, heart muscle cells, epithelial cells or various mucous membranes, hormone producing cells, and transmitter producing cells.

5. The method of claim 1, further comprising using the eucaryotic cells as an autologous transplant or preparing an autologous transplant or a three dimensional tissue substitution material from said eucaryotic cells.

6. The method of claim 2, further comprising using the eucaryotic cells as an autologous transplant or preparing an autologous transplant or a three dimensional tissue substitution material from said eucaryotic cells.

7. The method of claim 3, further comprising using the eucaryotic cells as an autologous transplant or preparing an autologous transplant or a three dimensional tissue substitution material from said eucaryotic cells.

8. The method of claim 4, further comprising using the eucaryotic cells as an autologous transplant or preparing an autologous transplant or a three dimensional tissue substitution material from said eucaryotic cells.

9. The method of claim 5, wherein said autologous transplant comprises skin cells, cells of the oral mucous membrane, cartilage cells, bone cells or adipocytes or a mixture of two or more thereof wherein said cells are in a matrix based upon biological molecules.

10. The method of claim 6, wherein said autologous transplant comprises skin cells, cells of the oral mucous membrane, cartilage cells, bone cells or adipocytes or a mixture of two or more thereof wherein said cells are in a matrix based upon biological molecules.

11. The method of claim 7, wherein said autologous transplant comprises skin cells, cells of the oral mucous membrane, cartilage cells, bone cells or adipocytes or a mixture of two or more thereof wherein said cells are in a matrix based upon biological molecules.

12. The method of claim 8, wherein said autologous transplant comprises skin cells, cells of the oral mucous membrane, cartilage cells, bone cells or adipocytes or a mixture of two or more thereof wherein said cells are in a matrix based upon biological molecules.

13. The method of claim 4, wherein said hormone producing cells or transmitter producing cells are cultivated with a capsule to form an allogenous transplant.

14. A transplant comprising melanocytes keratinocytes or a combination of melanocytes and keratinocytes wherein said melanocytes, said keratinocytes or said combination of melanocytes and keratinocytes are cultivated according to the cells of claim 1.

15. A composition suitable for transplanting comprising melanocytes in a carrier matrix.

16. The composition according to claim 15, wherein said carrier matrix exhibits plasticity or gel-like consistency.

17. The composition according to claim 16, wherein said carrier matrix comprises biomolecules, synthetic materials or a combination of both biomolecules and synthetic materials.

18. The composition according to claim 15, wherein the carrier matrix further comprises fibrin glue.

19. A method for treating vitiligo patients which comprises administering to a patient a composition according to claim 15 wherein said composition suitable for transplantation is an autologous transplant.

20. A method for treating a vitiligo patient with an autologous transplant comprising:
i) pretreating the patient with an ER-YAG-laser;
ii) transplanting a composition suitable for said autologous transplant comprising melanocytes in a carrier matrix wherein said melanocytes are cultivated according to the cells of claim 1; and
iii) posttreating the patient with UV-radiation.

21. A method for treating a vitiligo patient with an autologous transplant comprising:
i) pretreating the patient with an ER-YAG-laser;
ii) transplanting a composition suitable for said autologous transplant comprising melanocytes in a carrier matrix wherein said melanocytes are cultivated according to the cells of claim 2; and
iii) posttreating the patient with UV-radiation.

22. A method for treating a vitiligo patient with an autologous transplant comprising:
i) pretreating the patient with an ER-YAG-laser;
ii) transplanting a composition suitable for said autologous transplant comprising melanocytes in a carrier matrix wherein said melanocytes are cultivated according to the cells of claim 3; and
iii) posttreating the patient with UV-radiation.

23. A method for treating a vitiligo patient with an autologous transplant comprising:
i) pretreating the patient with an ER-YAG-laser;
ii) transplanting a composition suitable for said autologous transplant comprising melanocytes in a carrier matrix wherein said melanocytes are cultivated according to the cells of claim 4; and
iii) posttreating the patient with UV-radiation.

24. A method of treating a vitiligo patient comprising administering the autologous transplant or the three-dimensional substitution material of claim 5 to said patient.

25. A method of treating a vitiligo patient comprising administering the autologous transplant or the three-dimensional substitution material of claim 6 to said patient.

26. A method of treating a vitiligo patient comprising administering the autologous transplant or the three-dimensional substitution material of claim 7 to said patient.

27. A method of treating a vitiligo patient comprising administering the autologous transplant or the three-dimensional substitution material of claim 8 to said patient.

28. A method of treating a skin disorder comprising preparing an autologous transplant of keratinocytes and applying said autologous transplant to said skin disorder.

29. The method of claim 28 wherein said skin disorder is selected from the group consisting of (a) solar precancerous forms, (b) pregnancy-induced changes in skin, (c) rosacea and rhinophyn, (d) age-induced changes in skin, and (e) keloids, hypertrophic scares, acne scares and therapy resistant acne.

30. A method for treating a vitiligo patient with an autologous transplant comprising:
i) pretreating the patient with an ER-YAG-laser;
ii) transplanting said autologous transplant into said patient wherein said autologous transplant is prepared according to claim 9; and
iii) posttreating the patient with UV-radiation.

31. A method for treating a vitiligo patient with an autologous transplant comprising:
i) pretreating the patient with an ER-YAG-laser;
ii) transplanting said autologous transplant into said patient wherein said autologous transplant is prepared according to claim 10; and
iii) posttreating the patient with UW-radiation.

32. A method for treating a vitiligo patient with an autologous transplant comprising:
i) pretreating the patient with an ER-YAG-laser;
ii) transplanting said autologous transplant into said patient wherein said autologous transplant is prepared according to claim 11; and
iii) posttreating the patient with UV-radiation.

33. A method for treating a vitiligo patient with an autologous transplant comprising:
i) pretreating the patient with an ER-YAG-laser;
ii) transplanting said autologous transplant into said patient wherein said autologous transplant is prepared according to claim 12; and
iii) posttreating the patient with UV-radiation.

34. The method of claim 9, further comprising extracellular matrix molecules, derivatives of extracellular matrix molecules or synthetic molecules.

35. The method of claim 10, further comprising extracellular matrix molecules, derivatives of extracellular matrix molecules or synthetic molecules.

36. The method of claim 11, further comprising extracellular matrix molecules, derivatives of extracellular matrix molecules or synthetic molecules.

37. The method of claim 12, further comprising extracellular matrix molecules, derivatives of extracellular matrix molecules or synthetic molecules.

38. A method of using a composition suitable for transplantation comprising keratinocytes cultured according to claim 1 as an autologous transplant or for the preparation of an autologous transplant for the treatment of vitiligo patients wherein said cells are keratinocytes.

39. The method of claim 1 wherein said conditions comprise:
i) not more than 3,500 particles having a size of at least 0.5 mm present per cubic meter of air; and
ii) no particles have a size of at least 5 mm present per cubic meter of air.
